(19) United States
(12) Patent Application Publication
(10) Pub. No.: US 2025/0249206 A1
(43) Pub. Date: Aug. 7, 2025

(54) CATHETER FOR USE IN PAIN THERAPY

(71) Applicant: B. Braun Melsungen AG, Melsungen (DE)

(72) Inventor: Jens Wildhagen, Hannover (DE)

(21) Appl. No.: 19/044,808

(22) Filed: Feb. 4, 2025

(30) Foreign Application Priority Data
Feb. 6, 2024 (DE) 10 2024 103 223.5

(51) Int. Cl. A61M 25/00; A61N 1/05

(52) U.S. Cl. CPC A61M 25/0054; A61N 1/0551; A61M 2025/0063

(57) ABSTRACT

A catheter for use in pain therapy has a catheter shaft with a proximal end and a distal end. A catheter tip is arranged at the distal end of the catheter shaft, and a catheter hub is arranged at the proximal end of the catheter shaft. The catheter shaft has a pretensioning section that is elastically deformable between a pretensioning state and a compensating state. The pretensioning section is axially elastically compressed in the pretensioning state and has a first length. In the compensating state, the pretensioning section is not axially compressed or is less axially compressed and has a greater, second length, as a result of which, when a proximal dislocation of the catheter hub occurs, a proximal dislocation of the catheter tip is avoided.

### CROSS-REFERENCE TO RELATED APPLICATION

This application claims priority under 35 U.S.C. § 119 to German Application No. 10 2024 103 223.5, filed on Feb. 6, 2024, the content of which is incorporated by reference herein in its entirety.

### FIELD

The present disclosure relates to a catheter for use in pain therapy, comprising a catheter shaft, which extends between a proximal end and a distal end, a catheter tip, which is arranged at the distal end, and a catheter hub, which is arranged at the proximal end.

### BACKGROUND

Catheters are generally known in the field of medical engineering and are provided for use in pain therapy. In pain therapy called peripheral nerve block (PNB), the catheter is advanced distally with the aid of an insertion aid until the catheter tip is placed directly on the nerve that is to be anaesthetized. Cannulas and/or capillaries are used as insertion aids, it being possible in principle to make a distinction between different attachment techniques (“cannula over the needle”, “cannula through the needle”, “cannula through the capillary”). In some pain therapies, the catheter is used to administer a local anaesthetic. To anaesthetize the nerve, the local anaesthetic is dispensed via one or more catheter outlets located in the region of the catheter tip. In other pain therapies, the catheter is used for electrical neurostimulation. Electrical neurostimulation (in short, neurostimulation or neuromodulation) provides for the delivery of current pulses in the immediate vicinity of a nerve in order to influence the conduction behaviour of the latter for pain pulses.

### SUMMARY

The object of the present disclosure is to make available a catheter of the type mentioned at the outset that allows improved pain therapy.

This object is achieved in that the catheter shaft has a pretensioning section which is elastically deformable between a pretensioning state and a compensating state. In the pretensioning state, the pretensioning section is axially elastically compressed and has a first length. In the compensating state, the pretensioning section is not axially compressed or is less axially compressed and has a greater, second length. Between the pretensioning state and the compensating state, the pretensioning section performs a compensating movement brought about by the elastic pretensioning, by means of which a proximal dislocation of the catheter tip can be avoided and/or compensated in the event of a proximal dislocation of the catheter hub. The present disclosure is based on the finding that, when the catheter is used to administer a local anaesthetic, unintentional retraction of the catheter tip (proximal dislocation) can lead to insufficient flushing of the nerve with the local anaesthetic. When the catheter is used for neurostimulation, a proximal dislocation can lead to suboptimal stimulation of the nerve. In both treatment methods, this can adversely affect the efficacy of the pain therapy. Such proximal dislocation of the catheter tip is avoided by the solution according to the present disclosure. For this purpose, the catheter shaft comprises said pretensioning section. The pretensioning section can be compressed by exerting an external axial compressive load. In this case, the axial length of the pretensioning section shortens, hence at the same time also an axial length of the catheter shaft. This state is referred to as the pretensioning state. In the latter, the pretensioning section has said (compressed) first length. In the pretensioning state, the pretensioning section pretensions the distal end and the proximal end, such that the two ends are pretensioned axially away from each other. The pretensioning section is compressed to such an extent that its elastic pretension and the external compressive load compensate each other. Compressive load is understood to mean an axial exertion of force on the pretensioning section, from both sides of the pretensioning section and axially towards each other. As soon as the compressive load is reduced or completely removed, the pretensioning section stretches elastically until there is once again an equilibrium between the external compressive load and the pretensioning. This state is referred to as the compensating state. In the compensating state, the pretensioning section has said axial second length, which is greater than the first length. As a result of the elastic compensating movement of the pretensioning section during the transition from the pretensioning state to the compensating state, the pretensioning section compensates for a proximal dislocation of the catheter hub, with the result that there is no or at least a smaller proximal dislocation of the catheter tip.

In the solution according to the present disclosure, the elastic deformability of the pretensioning section has the effect that, when the compressive load is removed or reduced, the pretensioning section automatically deforms from the pretensioning state to the compensating state, i.e. without any further external force action besides the pressure relief. As a result, there is in particular no need for a tensile force which acts on the pretensioning section or on the proximal end or the catheter hub and causes the axial lengthening of the pretensioning section. In advantageous embodiments, the elastic deformability is a purely elastic deformability without in practice any considerable plastic or elastic-plastic deformation components. The catheter according to the present disclosure is applied to the body in such a way that the catheter tip is arranged in the body tissue close to a nerve to be anaesthetized and the pretensioning section is present in the pretensioning state. The pretensioning section pretensions the catheter tip in the distal direction, so that the catheter tip presses axially against the body tissue. The body tissue exerts an axial reaction force (compressive load) on the catheter tip and thereby holds the pretensioning section in the pretensioning state. In the event of a proximal dislocation of the catheter hub, the catheter tip initially also moves proximally by an infinitesimal path length. This reduces the axial reaction force. As a result, the distal pretensioning, which the pretensioning section exerts on the catheter tip in the distal direction, exceeds the axially oppositely directed compressive load. As a result, the pretensioning section stretches until there is an equilibrium of forces between the pretensioning force and the compressive load. On account of the increase in length of the catheter shaft in the region of the pretensioning section, there is a correspondingly reduced proximal dislocation or, in the best case, no proximal dislocation of the catheter tip. The pretensioning section can also be referred to as elastically deformable and/or elastically compressible. The increase in length of the pretensioning section can also be referred to as elongation.

In one embodiment of the present disclosure, the pretensioning section is produced from an elastically compressible material. The elastically compressible material is preferably an elastomer material. In this embodiment of the present disclosure, the catheter shaft is therefore produced from the elastically compressible material at least in the region of the pretensioning section, while the catheter shaft, away from the pretensioning section, is produced from a less easily compressible material. If the pretensioning section takes up the entire length of the catheter shaft, then the entire catheter shaft is produced from said elastically compressible material.

In a further embodiment of the present disclosure, the pretensioning section has a spring. The spring is preferably a helical spring.

In a further embodiment of the present disclosure, the pretensioning section has an elastically compressible configuration. In this embodiment of the present disclosure, the elastic deformability of the pretensioning section is not necessarily achieved through a suitable choice of material, but by a corresponding configuration. The increase in length or reduction in length takes place on account of a change in shape of the pretensioning section, for example on account of an accordion movement. In this embodiment of the present disclosure, the catheter shaft therefore has said elastically compressible configuration at least in the region of the pretensioning section, while the catheter shaft, away from the pretensioning section, has a different and less easily compressible configuration, preferably a smooth-walled cylindrical configuration known from the prior art. If the pretensioning section takes up the entire length of the catheter shaft, then the entire catheter shaft has said elastically compressible configuration.

In a further embodiment of the present disclosure, the elastically compressible configuration is an accordion-like configuration. In this embodiment, the pretensioning section is configured in the manner of an accordion. As a result, the pretensioning section can be easily compressed in the event of a corresponding compressive load. The accordion-like configuration can be formed in particular by a folded or undulating or helical design of the pretensioning section.

In a further embodiment of the present disclosure, the pretensioning section extends over an entire length of the catheter shaft. Consequently, in this embodiment of the present disclosure, the entire catheter shaft is elastically compressible. This embodiment of the present disclosure permits simplified production because the catheter shaft can be produced from one and the same material and/or can have one and the same design. This is in contrast to embodiments in which the pretensioning section occupies only a part of the total length of the catheter shaft.

In a further embodiment of the present disclosure, the pretensioning section extends over only a part of the total length of the catheter shaft, and the catheter shaft, away from the pretensioning section, has a compressive compliance which is less than a compressive compliance of the pretensioning section. Compliance is understood to mean the reciprocal of stiffness. For example, the catheter shaft can have, away from the pretensioning section, a compressive compliance which is at most 20%, preferably at most 5%, particularly preferably at most 1%, of the compressive compliance of the pretensioning section. As a result, the pretensioning section deforms elastically when a compressive load is exerted on the catheter shaft, whereas the catheter shaft away from the pretensioning section deforms elastically to a lesser extent or even not at all.

In a further embodiment of the present disclosure, the catheter shaft has a different configuration away from the pretensioning section. As a result of the different configurations of the pretensioning section and of the catheter shaft away from the pretensioning section, the pretensioning section can have a compressive compliance that is greater than a compressive compliance of the catheter shaft away from the pretensioning section.

In a further embodiment of the present disclosure, the catheter has a length-limiting device which is configured to limit the second length of the pretensioning section. The length-limiting device limits the second length of the pretensioning section to a predetermined value, which can be referred to as the limit length. The length-limiting device prevents stretching of the pretensioning section beyond the limit length. It is also conceivable that compression of the pretensioning section is possible by means of the length-limiting device.

In a further embodiment of the present disclosure, the length-limiting device has an elongate tension element, which bridges the pretensioning section to form a force relief. The tension element mechanically bridges the pretensioning section as soon as the latter reaches a maximum permissible length. When the maximum permissible length is reached, the tension element absorbs the compressive force applied by the pretensioning section and thereby forms said force relief, which can also be referred to as pressure relief. The tension element can be designed in any manner suitable for the present purpose. In some embodiments, the tension element is fastened on both axial sides of the pretensioning section, for example at the axial ends of the pretensioning section and/or at the distal end and/or at the proximal end of the catheter shaft.

In a further embodiment of the present disclosure, the elongate tension element has an electrically conductive wire, which is configured for the transmission and/or delivery of current pulses for neurostimulation. The wire thus has an advantageous multiple function. Firstly, the wire acts as a force relief. Secondly, the wire additionally functions as an electrical conductor for said neurostimulation. In this embodiment of the present disclosure, the catheter shaft and/or the catheter tip has at least one stimulation electrode, which is electrically conductively contacted by means of the conductive wire or is formed by the wire.

In a further embodiment of the present disclosure, the catheter has a holding device which is configured to secure the pretensioning section against deformation in the pretensioning state and to enable a deformation in the direction of the compensating state when activated. By means of the activation, it is possible to determine the time at which a possible elongation of the pretensioning section is permitted or enabled. For example, when the catheter is in place, the holding device can fix the pretensioning section in the pretensioning state. This prevents the pretensioning section from transitioning to the compensating state even before the final positioning of the catheter tip close to the nerve that is to be anaesthetized. In particular, the catheter can be applied in such a way that the catheter is advanced with the catheter tip in the direction of a nerve, while the holding device holds the pretensioning section in the pretensioning state and prevents its deformation. After the final position of the catheter tip is reached, the holding device can be activated, as a result of which a deformation of the pretensioning section is enabled and is only then possible. In some configurations, it may be possible to deactivate the holding device again after activation, such that the pretensioning section is held in the state in which it is located upon deactivation of the holding device, such that no further stretching and/or compression of the pretensioning section can take place. In some embodiments, the length-limiting device and the holding device are formed by one and the same component, which performs both functions. The activation can also be called an operation.

In some embodiments, the pretensioning section is in the compensating state when the catheter is applied. The body tissue to be penetrated when the catheter tip is advanced towards the nerve to be anaesthetized exerts an axial compressive force on the catheter tip, such that the pretensioning section is compressed. As soon as the catheter tip is arranged in the vicinity of the nerve, the pretensioning section can then be in the pretensioning state.

In a further embodiment of the present disclosure, the catheter has a support structure which secures the pretensioning section against a buckling movement in a direction transverse to the longitudinal extent. The support structure can ensure that the pretensioning section reliably retains its elastic deformability, preferably by virtue of the support structure radially stabilizing the pretensioning section. In advantageous embodiments, the support structure completely surrounds the pretensioning section in the circumferential direction. In some configurations, the support structure extends over a part of the pretensioning section such that another part of the pretensioning section is not surrounded by the support structure. In other configurations, the support structure extends over the entire length of the pretensioning section or even axially beyond the pretensioning section and over a part of the catheter shaft located away from the pretensioning section.

In a further embodiment of the present disclosure, the catheter shaft has a catheter lumen for administering a local anaesthetic. As a result, the local anaesthetic can be delivered directly to the surrounding body tissue via the catheter. Preferably, at least one catheter outlet is present in the region of the catheter tip, via which outlet the local anaesthetic can be delivered to the body tissue. In some embodiments, the catheter lumen extends through the pretensioning section, while in other embodiments the catheter lumen extends past the pretensioning section.

The present disclosure also relates to a catheter arrangement having a catheter according to the preceding description and having an insertion aid which extends between a distal end and a proximal end and has an insertion lumen for insertion of the catheter shaft. The insertion aid can also be called a capillary. In this embodiment, the catheter itself does not need to have a catheter lumen in order to be able to administer a local anaesthetic.

In a further embodiment of the present disclosure, with the catheter inserted into the insertion lumen, the insertion aid functions as a support structure for the pretensioning section. As a result, the insertion aid fulfils a multiple function. On the one hand, the insertion aid assists the placement of the catheter. On the other hand, the insertion aid secures the pretensioning section against a buckling movement in a direction transverse to the longitudinal extent.

### BRIEF DESCRIPTION OF THE DRAWING FIGURES

Further advantages and features of the present disclosure will emerge from the following description of preferred exemplary embodiments of the present disclosure, which are illustrated with the aid of the drawings.

FIG. 1 shows a schematic side view of an embodiment of a catheter according to the present disclosure, with a catheter shaft which has a pretensioning section in a pretensioning state,

FIG. 2 shows a further schematic side view of the catheter according to FIG. 1, with the pretensioning section in a compensating state,

FIGS. 3 and 4 show a schematic side view of an embodiment of a pretensioning section with an elastically compressible configuration, in a compressed state (FIG. 3) and in a non-compressed state (FIG. 4),

FIG. 5 shows a schematic side view of a further embodiment of a catheter according to the present disclosure, with the pretensioning section extending over an entire length of the catheter shaft,

FIG. 6 shows a schematic side view of a catheter arrangement having a catheter with a pretensioning section, which has a spring, and an insertion aid, wherein the insertion aid is shown in longitudinal section,

FIG. 7 shows a schematic side view of a further embodiment of a catheter according to the present disclosure, having a length-limiting device and a holding device.

### DETAILED DESCRIPTION

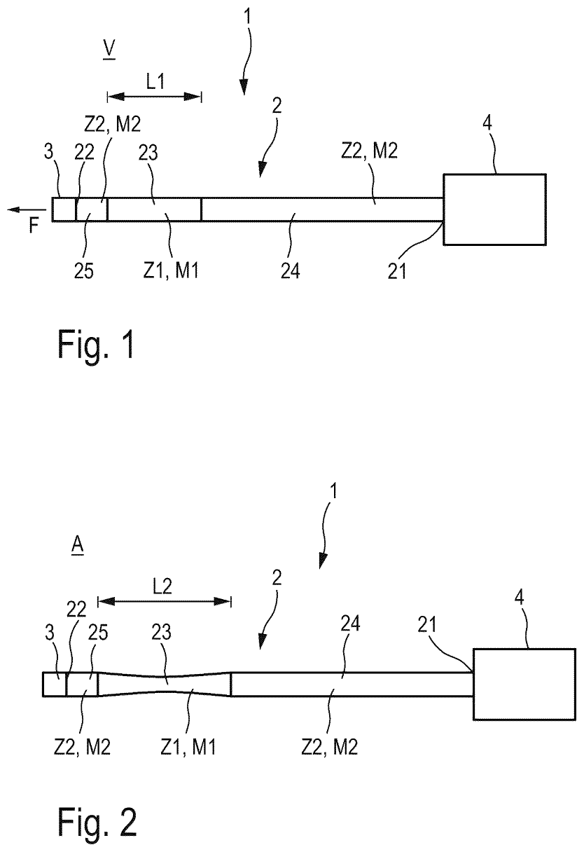

According to FIG. 1, a catheter 1 is provided for use in pain therapy, which is also referred to as peripheral nerve block. The catheter 1 can also be referred to as a pain catheter.

The catheter 1 has a catheter shaft 2, a catheter tip 3 and a catheter hub 4.

The catheter shaft 2 extends between a proximal end 21 and a distal end 22.

The catheter tip 3 is arranged at the distal end 22 of the catheter shaft 2. In the embodiment shown, the catheter tip 3 is produced separately from the catheter shaft 2 and is firmly connected to the distal end 22 of the latter. Alternatively, the catheter tip 3 can be an integral part of the catheter shaft 2 and form the distal end 22 thereof.

The catheter hub 4 is arranged at the proximal end 21 of the catheter shaft 2. The catheter hub 4 is firmly connected to the proximal end 21.

Pain therapy may comprise administration of a local anaesthetic. It will be appreciated that in this case the catheter 1 has at least one catheter lumen extending through the catheter shaft 2 and the catheter hub 4, and at least one catheter outlet connected fluidically to the catheter lumen for the purpose of dispensing the local anaesthetic. The at least one catheter outlet and the catheter lumen are not shown in any detail here. The catheter outlet or the catheter outlets are preferably arranged in the region of the catheter tip 3. In this case, the at least one catheter outlet can extend axially through the catheter tip 3 and/or radially through the catheter shaft 2. The number and arrangement of the catheter outlets is not essential as regards the present disclosure, and therefore further details are not explained. In other embodiments, the catheter 1 is used for electrical neurostimulation. For this purpose, the catheter 1 can have one or more electrodes. This will be explained in detail below.

For administration of the local anaesthetic or for neurostimulation, the catheter 1 is applied in a manner known to a person skilled in the art and is advanced distally until the catheter tip 3 is placed in immediate proximity to a nerve that is to be anaesthetized. The catheter 1 is preferably applied using an insertion aid, which will be explained in more detail below.

If, during the pain therapy, there is an unintended proximal movement of the catheter tip 3, referred to as (proximal) dislocation, the nerve to be anaesthetized, in the case of administration of a local anaesthetic, is no longer sufficiently surrounded by the local anaesthetic, or, in the case of electrical neurostimulation, is stimulated only suboptimally. This can adversely affect the efficacy of the pain therapy. To counteract this, the catheter shaft 2 has a pretensioning section 23.

The pretensioning section 23 is elastically deformable between a pretensioning state V and a compensating state A. In the pretensioning state V, the pretensioning section 23 is axially elastically compressed and has a first length L1. In the compensating state A, the pretensioning section 23 is not axially compressed or is less axially compressed and has a second length L2. The second length L2 is greater than the first length L1. As a result, in the event of a proximal dislocation of the catheter hub 4, a proximal dislocation of the catheter tip 3 can be avoided or at least reduced.

When the catheter 1 is applied and the catheter tip 3 is placed in immediate proximity to the nerve that is to be anaesthetized, the pretensioning section 23 pretensions the distal end 22 in the distal direction and the proximal end 21 in the proximal direction. As a result, the distal end 22 with the catheter tip 3 presses distally with a pretensioning force F against the body tissue surrounding the nerve that is to be anaesthetized. Accordingly, the body tissue surrounding the catheter tip 3 exerts an equally strong compressive force, i.e. an axial reaction force, directed counter to the pretensioning force F and not illustrated in any detail, on the catheter tip 3 and, via the distal end 22, on the pretensioning section 23. The pretensioning section 23 is thereby held in the pretensioning state V and maintains its first length L1. If, in this state, a proximal dislocation of the catheter hub 4 takes place, the catheter tip 3 moves proximally by an infinitesimal path length. The axial compressive force which the body tissue exerts on the catheter tip 3, and which counteracts the pretensioning force F, thereby reduces or even becomes zero. As a result, the pretensioning section 23 stretches until the catheter tip 3 again bears against the distally located body tissue and there is once again an equilibrium of forces between the pretensioning force F and the compressive force that is exerted on the catheter tip 3 by the body tissue. In this way, by means of the pretensioning section 23, a proximal dislocation of the catheter tip 3 can be avoided in the event of a proximal dislocation of the catheter hub 4.

FIG. 2 shows the pretensioning section 23 in the compensating state A, in which the pretensioning section 23 has the second length L2. The second length L2 is greater than the first length L1. The difference between the second length L2 and the first length L1 corresponds approximately or exactly to the proximal dislocation of the catheter hub 4. In the compensating state A, by changing its length, the pretensioning section 23 compensates for the proximal dislocation of the catheter hub 4. In the exemplary embodiment shown, the compensating state A corresponds to a rest state of the pretensioning section 23, in which no external axial forces act on the pretensioning section 23. Accordingly, the pretensioning section 23 itself does not exert a pretensioning force F on the distal end 22. In the exemplary embodiment shown in FIG. 2, the pretensioning section 23 cannot stretch further without the action of an external force, that is to say by itself. In other embodiments, the pretensioning section 23 also exerts a pretensioning force F on the surrounding body tissue in the compensating state A, which pretensioning force is lower than the pretensioning force F in the pretensioning state V. In the event of a further reduction of the compressive force acting axially on the catheter tip 3 through the body tissue, the pretensioning section 23 could stretch even further. Accordingly, the second length L2 of the pretensioning section 23 in the compensating state A is less than a maximum length of the pretensioning section 23. In the embodiment shown in FIGS. 1 and 2, the pretensioning section 23 extends over only a part of a total length of the catheter shaft 2. The arrangement of the pretensioning section 23 and its proportion of the total length are to be understood as examples.

In the present case, the pretensioning section 23 is arranged in the region of the distal end 22 of the catheter shaft 2. The catheter shaft 2 further comprises a proximal shaft section 24 and a distal shaft section 25. The proximal shaft section 24 is arranged axially between the proximal end 21 and the pretensioning section 23. The proximal shaft section 24 in the present case comprises the proximal end 21 of the catheter shaft 2 and is firmly connected at one end to the catheter hub 4. At the other end, the proximal shaft section 24 is firmly connected to the pretensioning section 23. The distal shaft section 25 is arranged axially between the catheter tip 3 and the pretensioning section 23. The distal shaft section 25 in the present case comprises the distal shaft end 22 and is firmly connected at one end to the catheter tip 3. At the other end, the distal shaft section 25 is firmly connected to the pretensioning section 23.

In an embodiment not shown in the figures, there is no distal shaft section 25, and instead the pretensioning section 23 is connected directly to the catheter tip 3.

The proximal shaft section 24 and the distal shaft section 25 are less easily axially deformable than the pretensioning section 23. In other words, the pretensioning section 23 has a greater compressive compliance (lower spring stiffness) than the proximal shaft section 24 and the distal shaft section 25. Conversely, the proximal shaft section 24 and the distal shaft section 25 have a lower compressive compliance (greater spring stiffness) than the pretensioning section 23. The different configuration in terms of the elastic deformability can be achieved through a correspondingly different choice of material and/or design and/or by provision of a spring and/or a compressible gas or liquid container or the like.

In the embodiment shown in FIGS. 1 and 2, the pretensioning section 23 has a compressive compliance Z1. Away from the pretensioning section 23, i.e. in the region of the proximal shaft section 24 and the distal shaft section 25, the catheter shaft 2 has a lower compressive compliance Z2. The two compressive compliances Z1, Z2 can also be referred to as first compressive compliance Z1 and second compressive compliance Z2. In other words, owing to the different materials, the pretensioning section 23 is elastically compressible, whereas the catheter shaft 2 is rigid or at least comparatively stiff or rigid away from the pretensioning section 23.

The second compressive compliance Z2 of the catheter shaft 2 away from the pretensioning section 23 is only 2% of the first compressive compliance Z1 in the present case. In embodiments not shown in the figures, the second compressive compliance is a maximum of 20%, preferably a maximum of 5%, of the first compressive compliance Z1.

In the embodiment according to FIGS. 1 and 2, the pretensioning section 23 is produced from a material M1. Away from the pretensioning section 23, i.e. in the region of the proximal shaft section 24 and of the distal shaft section 25, the catheter shaft 2 is produced from a different material M2. The two materials M1, M2 can also be referred to as first material M1 and second material M2.

Both materials M1, M2 in the present case are plastic materials suitable for medical uses. The materials M1, M2 are different at least in terms of their compressive compliance and/or spring stiffness.

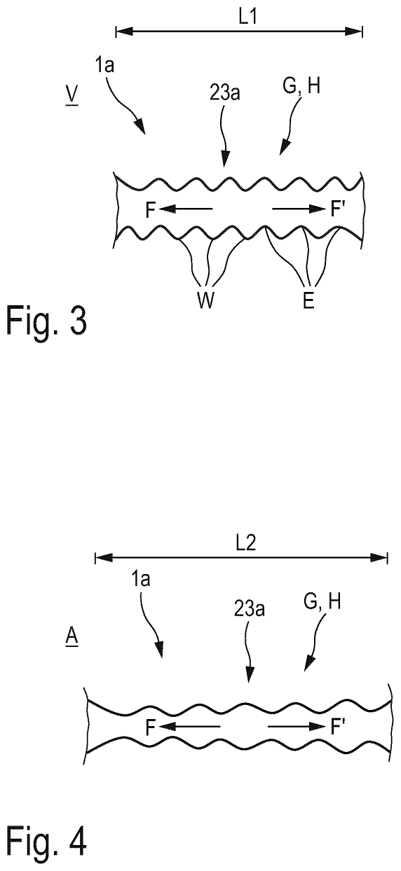

In an embodiment according to FIGS. 1 and 2, the elastic deformability of the pretensioning section 23 is thus achieved through a corresponding choice of material. Alternatively or additionally, the pretensioning section 23 can have an elastically compressible design. An elastically compressible design is shown by way of example in FIGS. 3 and 4.

FIGS. 3 and 4 show an embodiment of a catheter la in which the pretensioning section 23a has an elastically compressible design G. In particular, the pretensioning section 23a is designed in the manner of an accordion and in this sense has an accordion-like design H. The catheter shaft 2 is therefore provided, in the region of the pretensioning section 23, with radial protuberances W and radial indentations E. The protuberances W and the indentations E alternate in the axial direction and form said accordion-like design H.

FIG. 3 shows the pretensioning section 23a in the pretensioning state V. FIG. 4 shows the pretensioning section 23a in the compensating state A. In the pretensioning state V, the pretensioning section 23a exerts the distal pretensioning force F in the direction of the body tissue located distally of the catheter tip and an oppositely directed, proximal pretensioning force F′ of equal magnitude and directed away from the distal pretensioning force F in the direction of the catheter hub. In the event of a proximal dislocation, the distal pretensioning force F is no longer counteracted by a proximal compressive force of equal magnitude, such that the accordion-like design H stretches axially to such an extent that the proximal compressive force exerted on the catheter tip 3 by the body tissue compensates again for the distal pretensioning force F. The pretensioning force F is lower in the compensating state A according to FIG. 4 than in the pretensioning state V according to FIG. 3, which is illustrated graphically by a shorter force arrow. In the same way, the proximal pretensioning force F′ also decreases between the pretensioning state V and the compensating state A. In the compensating state A, the accordion-like design H has compensated for a proximal dislocation with a length which corresponds to the difference between the second length L2 of the accordion-like design H in the compensating state A and the first length L1 of the accordion-like design H in the pretensioning state V.

It will be appreciated that the number, shape and dimensioning of the protuberances W and of the indentations E shown in FIGS. 3 and 4 are to be understood purely as examples.

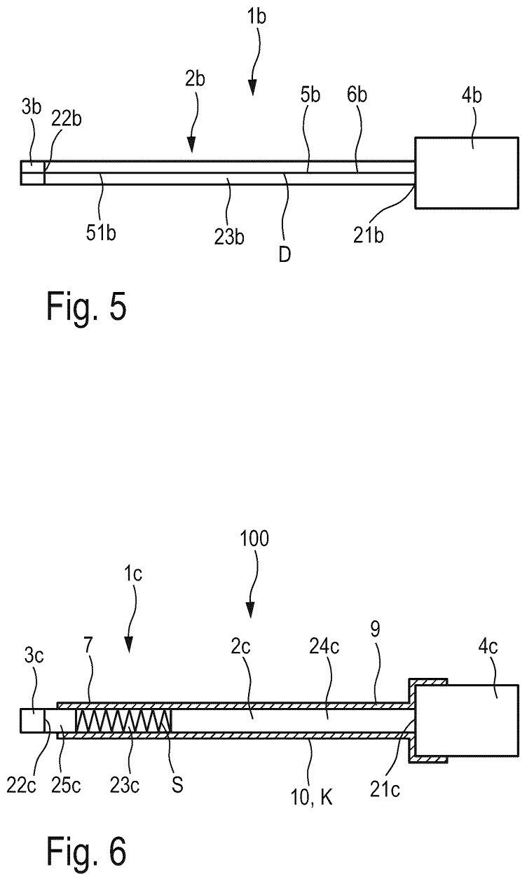

FIGS. 5 to 7 show further embodiments of catheters 1b, 1c, 1d according to the present disclosure. In order to avoid repetition, the main differences between the catheters 1b, 1c, 1d and the catheter 1 and the catheter 1a according to FIGS. 1 to 4 are discussed below. Functionally identical components and/or sections of the catheters 1b, 1c, 1d are provided with identical reference numbers with the addition of lower-case letters. Unless stated otherwise, the details that have been disclosed concerning the catheters 1, 1a according to FIGS. 1 to 4 also apply mutatis mutandis to the catheters 1b, 1c, 1d according to FIGS. 5 to 7.

In the case of the catheter 1b according to FIG. 5, the pretensioning section 23b extends over an entire length of the catheter shaft 2b. Thus, in comparison to the catheter 1 according to FIGS. 1 and 2, simpler production can be achieved. Moreover, the pretensioning section 23 can be elastically compressed over a greater length, as a result of which greater proximal dislocations of the catheter hub 4 can be compensated.

The catheter 1b has a length-limiting device 5b. The length-limiting device 5b is configured to limit the length or an increase in length of the pretensioning section 23b. The length-limiting device 5b can be designed differently in the case of different configurations.

In the embodiment of FIG. 5, the length-limiting device 5b has a tension element 51b which bridges the pretensioning section 23b to form a force relief. As soon as the pretensioning section 23b reaches a maximum permissible second length L2 in the compensating state A, the length-limiting device 5b acts as a force relief D and effects a force compensation of the pretensioning force F internal to the catheter shaft, such that the pretensioning section 23b cannot extend further. In this state, a proximal dislocation of the catheter hub 4b can lead directly to a proximal dislocation of the catheter tip 3b. By way of example, removal of the catheter 1b from the body tissue can be facilitated when the pretensioning section 23b is relieved of force by means of the tension element 51b.

Since, in the embodiment shown in FIG. 5, the pretensioning section 23b extends over the entire length of the catheter shaft 2b, the tension element 51b correspondingly also extends over the entire length of the catheter shaft 2b. In other embodiments (not shown), in which the pretensioning section extends only over part of the overall length of the catheter shaft, the tension element can also extend only over the length of the pretensioning section or alternatively over the entire length of the catheter shaft.

In the embodiment shown in FIG. 5, a holding device 6b is additionally provided, which is configured to secure the pretensioning section 23b against a deformation in the pretensioning state V. The holding device 6b is activatable. Upon activation, the holding device 6b enables a deformation of the pretensioning section 23b. This means that deformation of the pretensioning section 23b is possible only after activation of the holding device 6b. In some embodiments, the holding device 6b can be designed in such a way that, before activation, it permits compression of the pretensioning section 23b, while it prevents stretching of the pretensioning section 23b. In other embodiments, the holding device 6b can be designed in such a way that, before activation, it does not permit either compression or stretching of the pretensioning section 23b.

The holding device 6b can facilitate application of the catheter 1b and a distal advance of the catheter tip 3b into the immediate vicinity of the nerve that is to be anaesthetized, since the holding device 6b is not activated during the advance, and therefore the catheter 1b can be advanced distally with unchanged or unchangeable length. After placement of the catheter tip 3b in the immediate vicinity of the nerve that is to be anaesthetized, the holding device 6b is activated, so that the pretensioning section 23b can compensate for a proximal dislocation.

In the embodiment shown, the tension element 51b also functions as a holding device 6b and accordingly fulfils both the length-limiting function and the release function.

In the embodiment shown in FIG. 6, the pretensioning section 23c extends over only a part of the entire length of the catheter shaft 2c. The pretensioning section 23c has a spring S. Specifically, the spring S is designed as a helical compression spring.

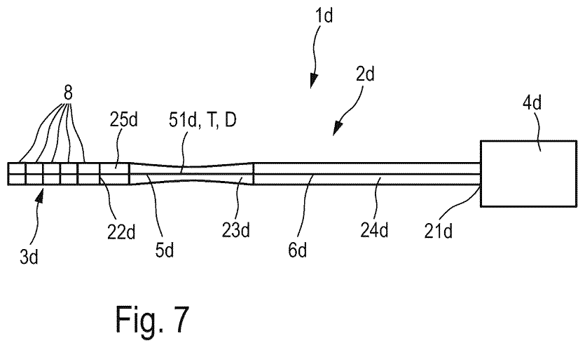

The catheter 1d shown in FIG. 7 is designed as a stimulation catheter for electrical neurostimulation. For this purpose, the catheter 1d has a plurality of stimulation electrodes 8 at the catheter tip 3d; specifically, five stimulation electrodes 8 are shown. By way of the stimulation electrodes 8, the catheter 1d can emit electrical current pulses to the body tissue surrounding the catheter tip 3d. The stimulation electrodes 8 are connected to the catheter hub 4d via an electrically conductive wire T. The wire T thus extends from the catheter tip 3d over the entire length of the catheter shaft 2d to the catheter hub 4d. In particular, the wire T extends over the entire length of the pretensioning section 23d, which is adjoined distally by a distal shaft section 25d and proximally by a proximal shaft section 24d. The wire T is configured to transmit current pulses for neurostimulation of the surrounding body tissue. Furthermore, the wire T functions as a tension element 51d, which can bridge the pretensioning section 23d and form a force relief. Moreover, the electrically conductive wire T functions as a length-limiting device 5d for limiting the second length L2 of the pretensioning section 23d. In order to fulfil the function of the length-limiting device 5d and of the holding device 6d, the electrically conductive wire T can be connected to the pretensioning section 23d at the contact points in each case to the proximal shaft section 24d and the distal shaft section 25d.

In embodiments not shown, the catheter can be designed both for delivering a local anaesthetic and for electrical neurostimulation and accordingly can have a catheter lumen and at least one catheter outlet and also at least one stimulation electrode.

FIG. 6 shows a catheter arrangement 100 with the catheter 1c already described above and with an insertion aid 10. The insertion aid 10 is designed here as a capillary K. The insertion aid 10 has an insertion lumen 9, which is configured to receive the catheter shaft 2c. In the configuration shown in FIG. 6, the catheter shaft 2c is pushed into the insertion lumen 9 via a proximal end (not designated in any more detail) of the insertion aid 10, wherein a part of the distal end 22c of the catheter shaft 2c together with the catheter tip 3c projects distally out of the insertion lumen 9 of the insertion aid 10. The pretensioning section 23c is arranged completely, i.e. over its entire length, in the insertion lumen 9. The catheter hub 4c and the proximal end of the insertion aid 10 are preferably designed to form a releasable connection. The releasable connection can be a screw, Luer or other suitable connection for the present purpose.

The insertion aid 10 fulfils a multiple function in the embodiment shown in FIG. 6. On the one hand, it assists the placement of the catheter 1c. On the other hand, the insertion aid 10 functions as a support structure 7 which secures the pretensioning section 23c against a buckling movement in a direction transverse to the longitudinal extent of the catheter shaft 2c. By virtue of the fact that the insertion aid 10 surrounds the spring S over the entire length and over the entire circumference, the pressure-loaded spring S cannot yield or buckle to the side. This ensures reliable functioning of the spring S. Moreover, the insertion aid 10 can serve as protection for the pretensioning section 23c against external influences and can thereby facilitate, for example, transport of the catheter arrangement 100.

In addition, it is conceivable that a local anaesthetic is delivered via the insertion lumen and that current pulses are delivered to the surrounding body tissue via a stimulation catheter arranged in the insertion lumen. In this case, the local anaesthetic can be guided to the distal end of the insertion aid, for example, through the gap between the insertion aid and the stimulation catheter.

In an alternative embodiment (not shown), a separate support structure is provided which secures the pretensioning section against a buckling movement. The support structure can be fastened, for example, to the distal shaft section and/or to the proximal shaft section and/or to the pretensioning section.

It will be appreciated that individual features of the catheters 1, 1a, 1b, 1c, 1d can be combined with one another to form further embodiments according to the present disclosure.

1. A catheter for use in pain therapy, the catheter comprising:
a catheter shaft having a proximal end and a distal end;
a catheter tip arranged at the distal end of the catheter shaft; and
a catheter hub arranged at the proximal end of the catheter shaft,
the catheter shaft having a pretensioning section that is elastically deformable between a pretensioning state and a compensating state,
the pretensioning section, when in the pretensioning state, being axially compressed and having a first length,
the pretensioning section, when in the compensating state, being axially compressed to a lesser extent than in the pretensioning state, and having a second length greater than the first length, such that, when a proximal dislocation of the catheter hub occurs, a proximal dislocation of the catheter tip is prevented.

2. The catheter according to claim 1, wherein the pretensioning section comprises a spring.

3. The catheter according to claim 1, wherein the pretensioning section comprises an accordion-like configuration.

4. The catheter according to claim 1, wherein the pretensioning section extends over an entire length of the catheter shaft.

5. The catheter according to claim 1, wherein the pretensioning section extends over only a part of a total length of the catheter shaft.

6. The catheter according to claim 5, wherein the catheter shaft, away from the pretensioning section, has a first compressive compliance, and wherein the pretensioning section has a second compressive compliance that is greater than the first compressive compliance.

7. The catheter according to claim 5, wherein the catheter shaft, away from the pretensioning section, has a different configuration and/or is produced from a different material than the pretensioning section.

8. The catheter according to claim 1, further comprising a length-limiting device configured to limit the second length of the pretensioning section.

9. The catheter according to claim 8, wherein the length-limiting device has an elongate tension element that bridges the pretensioning section to form a force relief.

10. The catheter according to claim 9, wherein the elongate tension element has an electrically conductive wire configured for transmission and/or delivery of current pulses for neurostimulation.

11. The catheter according to claim 1, further comprising a holding device configured to secure the pretensioning section against deformation in the pretensioning state and, upon activation, to allow deformation of the pretensioning section.

12. The catheter according to claim 1, further comprising a support structure that secures the pretensioning section against a buckling movement in a direction transverse to a longitudinal extent of the catheter shaft.

13. The catheter according to claim 1, further comprising a catheter lumen for administering a local anaesthetic.

14. A catheter arrangement comprising:
the catheter according to claim 1; and
an insertion aid having a proximal insertion aid end and a distal insertion aid end,
the insertion aid comprising an insertion lumen for insertion of the catheter shaft.

15. The catheter arrangement according to claim 14, wherein the insertion aid forms a support structure for the pretensioning section when the catheter shaft is inserted into the insertion lumen.
